(19) United States
(12) Patent Application Publication
(10) Pub. No.: US 2007/0224297 A1
(43) Pub. Date: Sep. 27, 2007

(54) Method for increasing content of physiologically active substance in ginseng

(76) Inventors: Deok Hoon Park, Yongin-si (KR); Jong Sung Lee, Anyang-si (KR); Eunsun Jung, Suwon-si (KR)

Correspondence Address: O'KEEFE, EGAN, PETERMAN & ENDERS LLP, 1101 CAPITAL OF TEXAS HIGHWAY SOUTH, #C200, AUSTIN, TX, 78746, US

(21) Appl. No.: 11/725,885

(22) Filed: Mar. 20, 2007

(30) Foreign Application Priority Data
Mar. 21, 2006 (KR) 10-2006-0025865

(51) Int. Cl. A61K 36/258; A61K 8/97

(52) U.S. Cl. 424/728; 424/74

(57) ABSTRACT

The present invention relates to a method for economically preparing ginseng extract having an increased content of physiologically active substances, comprising the steps of steaming ginseng at 95 to 100° C. for 1 to 24 hours, collecting the generated vapor by cooling, and repeating the process twice, to a composition for promoting collagen synthesis, comprising the extract, and to a cosmetic product for improving skin wrinkles, comprising the composition.

### BACKGROUND OF THE INVENTION

1. Field of the Invention

The present invention relates to a method for increasing the content of the physiologically active ingredients present in ginseng (Panax ginseng), and more specifically to a method for increasing the content of Rb1, Rb2, Rc and Rd, which are trace amounts of Panax Diol-based ginseng saponins present in ginseng.

2. Description of the Related Art

The ginseng has been used as a medical plant which replenishes and strengthens blood in Oriental countries, including Korea and China. Although there exist various types of plants in the United States, Japan, China, Russia, and the like, which are similar to Korean ginseng, but they are different from the Korean ginseng produced in Korea in terms of the family, type and components. Among the all the different types of ginseng, the Korean ginseng is considered to be most preferred. The Korean ginseng belongs to genus ginseng of Araliaceae Panax, which has a common plant name of Panax ginseng C. A. Meyer. The genus Panax is divided into six categories, which are Korean ginseng (P. ginseng), American ginseng (P. quinquefolium), Panax notoginseng Burkillr (P. notoginseng), Japanese ginseng (P. japonicum), Sanchi ginseng (P. trifolium) and Himalayan ginseng (P. Pseudoginseng).

The types of ginseng saponins (ginsenoside, G) contained in the Korean ginseng are divided into the following three categories:

19 kinds of protopanaxadiol (PD)-based saponins (G-Ra1, G-Ra2, G-Ra3, G-Rb1, G-Rb2, G-Rb3, G-Rc, G-Rd, 20(S)G-Rg3, 20(R)G-Rg3, G-Rh2, G-Rsl, G-Rs2, Quinquenoside-R1, Notoginsenoside-R4, Malonyl-G-Rb1, Malonyl-G-Rb2, Malonyl-G-Rc, Malonyl-G-Rd), 10 kinds of protopanaxatriol (PT)-based saponins (G-Re, G-Rf, G-Rg1, G-Rg2, G-Rh1, 20-Glc-G-Rf, Notoginsenoside-R1, 20(R)G-Rg2, 20(R)G-Rh1, Rh2), and

1 kind of oleanane-based saponin (G-Ro).

A conventional method for preparing Red ginseng comprises the steps of steaming 6-year-old Fresh ginseng and drying it, wherein the Fresh ginseng is processed by repeating the steaming and drying step several times.

Korean Patent No. 10-0545324 discloses a preparation method, comprising the steps of: first, steaming ginseng at a temperature of 95 to 100° C. for 1 to 12 hours to increase the content of ginsenocide having an anticancer activity; second, drying ginseng at a temperature of 50 to 80° C., while retaining a moisture content of 70 to 80%; third, leaving the processed ginseng obtained from the secondary drying step at a temperature in the range of −10 to −30° C. for 10 to 30 hours to isolate the fiber tissues of the taproot; and finally, steaming the ginseng at a temperature of 100 to 120° C. for 2 to 12 hours, and then at a temperature of 50 to 80° C. for 1 to 5 hours. Further, Korean Patent No. 10-336086 discloses a method for preparing Red ginseng extract, comprising the steps of: steaming Fresh ginseng or White ginseng using vapor generated by heating water; drying the steamed ginseng; and heating the dried ginseng in a water bath to extract the effective ingredients.

Although the related art techniques simplified the whole procedures as compared with a conventional preparation of Red ginseng and increased the content of some of the ginsenocides, but a better method for increasing the ginsenocides and simplifing the process is needed. Therefore, the present inventors developed a better method to economically prepare the ginseng while reducing the number of processes, and they found that the ingredients produced by the method promote collagen formation, thereby completing the present invention.

### SUMMARY OF THE INVENTION

It is an object of the present invention to provide a method for preparing a ginseng extract having an increased content of physiologically active ingredients, comprising the steps of steaming ginseng at 95 to 100° C. for 1 to 24 hour, collecting the generated vapor by cooling, and repeating the process twice.

It is another object of the present invention to provide a composition for promoting collagen formation, comprising the extract as prepared above.

It is still another object of the present invention to provide a cosmetic product for improving skin wrinkles, comprising the composition for promoting collagen formation.

### BRIEF DESCRIPTION OF THE DRAWINGS

FIG. 1 illustrates the effect of activating the collagen promoter by the ginseng extract prepared using the method of the present invention (GE: ginseng extract, ETOH: ethanol).

FIG. 2 illustrates the effect of promoting collagen biosynthesis by the ginseng extract prepared using the method of the present invention (UA: ursolic acid, ETOH: ethanol).

FIG. 3 illustrates the effect of inhibiting collagenase activity by the ginseng extract prepared using the method of the present invention (TNF alpha: tumor necrosis factor alpha).

### DESCRIPTION OF THE PREFERRED EMBODIMENTS

In one embodiment, the present invention relates to a method for preparing a ginseng extract having an increased content of physiologically active ingredients, comprising the steps of steaming ginseng at 95 to 100° C. for 1 to 24 hour, collecting the generated vapor by cooling, and repeating the process twice.

The term “ginseng” as used herein encompasses plants belonging to the ginseng geni Araliaceae and Panax, including Fresh ginseng, Wild ginseng and Camphor ginseng. In specific embodiments of the present invention, unprocessed ginseng such as Fresh ginseng is used, and undried Fresh ginseng is preferably used.

The term, “physiologically active ingredients” means the ingredients contained in the ginseng, for example, Rb1, Rb2, Rc and Rd, as Panax Diol-based ginseng saponins, which have a central nerve inhibiting action, an anesthesia action, an analgesia action, a psychological security action, an antipyretic action, a hormone release promoting action, an antipyretic action and a neutral fat decomposition inhibiting action.

In specific embodiments of the present invention, the undried ginseng is steamed at 95 to 100° C. for 1 to 24 hours, and then the generated vapor is collected by cooling. Thereafter, this step is repeated one more time. Then, the extracts collected from each step are mixed, as a result, the ginseng extract having an increased content of the physiologically active ingredients according to the present invention can be prepared.

Unprocessed ginseng such as Fresh ginseng has a much lower content of ginseng saponins. However, by steaming the Fresh ginseng to be processed into Red ginseng, the content of the active substances is increased. A conventional method for preparing Red ginseng comprising the steps of ‘steaming’ and ‘cooling’ nine times, resulting in an extremely complicated process. One technique for further simplifying the steaming and cooling steps by reducing the repetition number of the process from nine to seven is disclosed in Korean Patent No. 10-529475. Korean Patent No. 10-545324 also discloses a method for increasing the content of the effective active substances by only repeating the steps of steaming and cooling twice, that is, in particular, steaming and cooling as a first step, and then steaming as a second step. In addition, the preparation method of the present invention can be further simplified by only performing “the steaming and cooling steps” twice, as compared with the above-described methods, thereby it being economical. Further, the preparation method of the present invention remarkably increased the content of Rb1, Rb2, Rc and Rd, which are Panax Diol-based ginseng saponins, by performing “the steaming and cooling step” twice, and collecting the generated vapor during steaming (Table 1).

In another embodiment, the present invention relates to a composition for promoting collagen synthesis, comprising the ginseng extract prepared by performing the steps of steaming ginseng at 95 to 100° C. for 1 to 24 hours, collecting the generated vapor by cooling, and repeating the process twice.

The term, “the composition for promoting collagen synthesis” as used herein means enhancing collagen synthesis, and at the same time, inhibiting the activity of collagenase as a collagen decomposing enzyme to increase collagen synthesis.

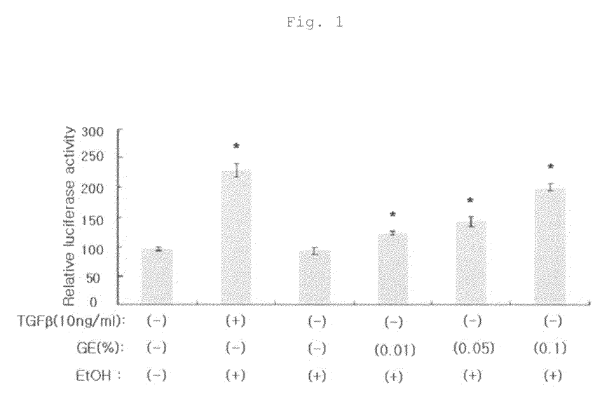

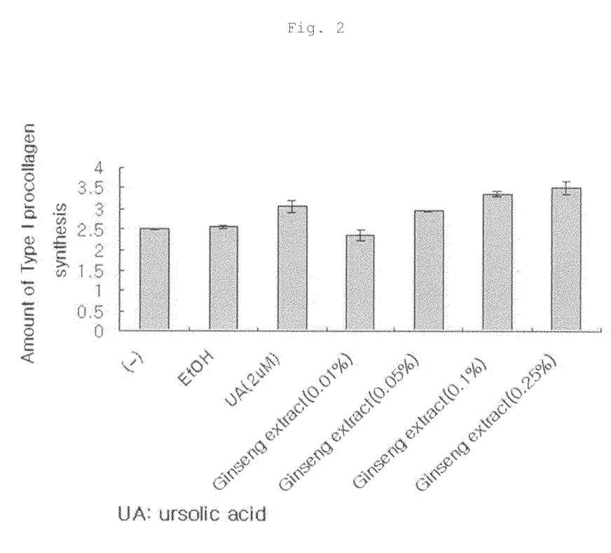

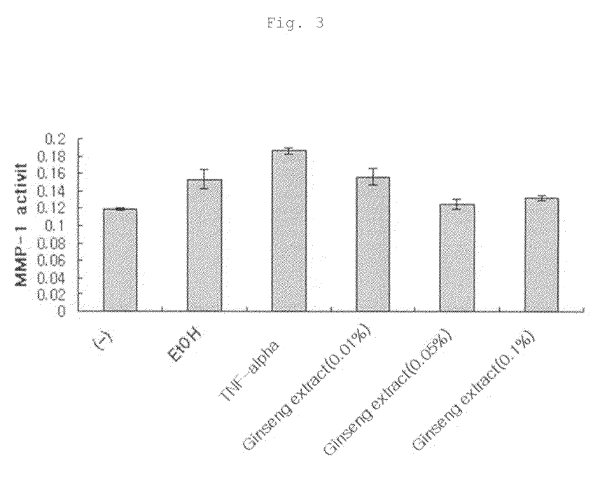

The ginseng extract prepared by the method of the present invention has an increased amount of Rb1, Rb2, Rc and Rd, which are Panax Diol-based ginseng saponins, as compared with the conventional ginseng extracts, and the ingredients containing them promote the physiological activity of a human body. Particularly, the ginseng extract of the present invention induced activation of the collagen promoter in a human normal fibroblast (see Example 2 and FIG. 1), as well as increased collagen I synthesis (see Example 3 and FIG. 2). Also, it exhibited an excellent effect of inhibiting collagenase activity as a collagen decomposing enzyme (see Example 4 and FIG. 3).

The ginseng extract of the present invention is contained in a proportion of 0.001 to 20.0 w/w, preferably 0.001 to 10.0 w/w, and more preferably 0.001 to 5.0 w/w, based on the total dry weight of a composition for promoting collagen synthesis.

The composition for promoting collagen synthesis of the present invention can further comprise other well-known substances for promoting collagen synthesis, in addition to the ginseng extract prepared by the method of the present invention. The substances for promoting collagen synthesis include, for example, asiatic acid, asiaticoside, and a polygonum cuspidatum root extract.

In another embodiment, the present invention relates to a cosmetic product for improving skin wrinkles, comprising the composition for promoting collagen synthesis.

The cosmetic product for improving skin wrinkles of the present invention contains the composition for promoting collagen synthesis in a proportion of 0.001 to 20.0 wt %, preferably 0.001 to 10.0 wt %, and most preferably 0.001 to 5.0 wt %, based on the total weight of the cosmetic product, but not limited thereto.

The ingredients contained in the cosmetic product of the present invention are effective ingredients, including commonly used ingredients in the cosmetic product, in addition to the composition. Such ingredients include, for example, conventional auxiliary agents such as an antioxidant, a stabilizer, a solubilizing agent, a vitamin, a pigment, and a flavor, and a carrier.

The cosmetic product of the present invention can be prepared as any formulation commonly prepared in the art. The cosmetic product can be formulated as, for example, a solution, a suspension, an emulsion, a paste, a gel, a cream, a lotion, a powder, a soap, a surfactant-containing cleanser, an oil, a powdered foundation, an emulsion foundation, a wax foundation, a spray, or the like, but not limited thereto. More specifically, the cosmetic product can be prepared as a formulation such as a softening toner, a nutrient toner, a nutrient cream, a massage cream, essence, an eye cream, a cleansing cream, a cleansing foam, a cleansing water, a pack, a spray, and a powder. In specific embodiments of the present invention, the cosmetic product is prepared as a nutrient cream, an astringent toner, a softening toner, an essence, a nutrient gel, a lotion, or a massage cream.

If the formulation of the cosmetic product of the present invention is a paste, a cream or a gel, an animal oil, a vegetable oil, a wax, paraffin, a starch, traganth, a cellulose derivative, a polyethylene glycol, silicone, bentonite, silica, talc, zinc oxide, or the like can be used as the carrier ingredient.

If the formulation of the cosmetic product of the present invention is a paste, a cream or a gel, lactose, talc, silica, aluminum hydroxide, calcium silicate, or polyamide powders can be used as the carrier ingredient, and in particular, if the formulation is a spray, a propellent such as chlorofluorohydrocarbon, propane/butane or dimethyl ether can be contained.

If the formulation of the cosmetic product of the present invention is a solution or an emulsion, a solvent, a solubilizing agent or an emulsifier can be used as the carrier ingredient, and examples thereof include water, ethanol, isopropanol, ethyl carbonate, ethyl acetate, benzyl alcohol, benzyl benzoate, propylene glycol, 1,3-butylglycol oil, glycerol aliphatic esters, polyethylene glycol or sorbitan fatty acid esters.

If the formulation of the cosmetic product of the present invention is a suspension, a liquid diluent such as water, ethanol and propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, traganth, or the like can be used as the carrier ingredient.

If the formulation of the cosmetic product of the present invention is a surfactant-containing cleanser, aliphatic alcohol sulfate, aliphatic alcohol ether sulfate, sulphosuccinic acid monoester, isethionate, imidazolinium derivative, methyltaurate, sarcosinate, fatty acid amide ether sulfate, alkylamidobetain, aliphatic alcohol, fatty acid glyceride, fatty acid diethanolamide, vegetable oils, a lanolin derivative or ethoxylated glycerol fatty acid ester, or the like can be used as the carrier ingredient.

The ginseng extract prepared according to the present invention has substantially no toxicity and/or side-effect (see Example 6 and Table 11), and has an excellent antioxidant effect (see Example 5), while also having skin wrinkles improving effect (see Example 7 and Table 12), whereby it can be used for a cosmetic product for improving skin wrinkles, comprising the composition for promoting collagen synthesis without negative or adverse affects.

Hereinbelow, the present invention will be described in more detail with reference to Examples. Examples are provided only for the purpose of illustrating the present invention, and accordingly it is not intended that the present invention is limited thereto.

### EXAMPLES

### Example 1

### Comparison of Extract Ingredients Prepared by Ginseng Extract Preparation Methods Between the Present Invention and the Related Art

### 1-1. Preparation of Ginseng Extract of the Present Invention

In order to prepare ginseng extract containing large amounts of Rb1, Rb2, Rc, etc. as the physiologically active ingredients, and increasing the total yield of ginsenocide from ginseng, 100 g of undried Fresh ginseng was weighed into a pressure steaming device, steamed at 95 to 100° C. for 1 to 24 hours, and cooled to collect the generated vapor. For a cooling method, the vapor traveling through the vapor tube was cooled using a condenser that is cooled by cooling water. This step is repeated one more time. Then, the extracts collected from each step are mixed, whereby ginseng extract of the present invention was prepared.

There were five different units of the device used for the preparation of the ginseng extract, and they are as follows:

A. Steam unit: Unit for generating steam and transferring the steam into a retort.

B. Retort unit: Unit, in which the vapor passes through a perforated floor and is brought into contact with a plant sample on a substrate, thereby generating the volatile ingredients in the water vapor form from the plant sample.

C. Vapor tube unit: Unit (tube) for transferring the vaporized water vapor into the condenser.

D. Condenser: Unit for converting the vaporized water vapor into the liquid state.

E. Separator: Unit for separating the liquid into an oil fraction and a water fraction.

### 1-2. Preparation of Ginseng Extract by Method of the Related Art (Comparative Examples 1 and 2)

### Comparative Example 1

An extract was prepared using the method disclosed in Korean Patent No. 10-0545324. In a pressure steaming sterilizer, ginseng was dried to have a moisture content of 50% in the Fresh ginseng state, and 100 g of the dried ginseng was weighed. Then, the ginseng was firstly steamed at 95 to 100° C. for 6 hours, and dried to have a moisture content of 75% by a hot air dryer maintained at 60° C. The dried ginseng processed firstly was left to stand in a freezer maintained at around −20° C. for 24 hours, to isolate the fiber tissues of the taproot. Thereafter, the resultant was secondarily steamed at a high temperature of 110° C. for 6 hours, and further steamed at around 70° C. for 2 hours to prepare a ginseng extract.

### Comparative Example 2

An extract was prepared using the method disclosed in Korean Patent No. 10-0529475. This is a procedure reduced the repetition number of the steaming and cooling steps from nine as in the conventional method for preparing Red ginseng to seven.

### 1-3. Comparison of Effective Ingredients of the Prepared Ginseng Extract

Analysis of the content of ginsenoside was conducted using a gas chromatography method and a high performance liquid chromatography method, and the condition of each method is shown as follows. The result of analysis is shown in Table 1.

### [Gas Chromatography Method]

Injector: split ratio 1:50

Detector: FID (Flame Ionization Detector)

Column: 30 m DBWAX 0.25 mm LD

Column pressure: 10 psi

Injector temperature: 250° C.

Detector temperature: 250° C.

Oven temperature program

Initial temperature: 200° C.

Heating rate: 4° C./min, up to 250° C.

### [High Performance Liquid Chromatography Method]

Apparatus: Dionex P530 pump, ASI-100 automated sample injector

column: Phenomenex Luna 5u (C18(2)), 150×4.6 mm

Mobile phase: 10 mM KH₂PO₄: water=92:8 (0-6.5 min)−40:60 (7.5-12 min)−92:8 (12.5-15 min) gradient system

Flow rate: 0.5 ml/min

Detector: UV/Vis Detector UVD 340S 260 nm

Injection volume: 10 ul

Operation time: 25 min

TABLE 1

| | | Comparative | Comparative |
|---|---|---|---|
| Ginsenosides | Example 1 (%) | Example 1 (%) | Example 2 (%) |
| Rb1 | 11.13 | 0.48 | 0.84 |
| Rb2 | 13.39 | 0.31 | 0.69 |
| Rc | 10.01 | 0.40 | 0.37 |
| Rd | 3.64 | 0.33 | 0.48 |
| Re | 1.05 | 0.23 | 0.74 |
| Rf | 2.16 | 2.68 | 0.66 |
| Rg1 | 0.35 | 3.83 | 1.04 |
| Rg2 | 2.25 | 2.04 | 0.97 |
| Rg3 | 1.16 | 3.75 | 1.06 |
| Total amount | 45.14 | 14.05 | 6.85 |

As shown in Table 1, it can be seen that when the effective ingredients are extracted from ginseng according to the present invention, the total content of ginsenoside in the ginseng extract prepared by the method of the present invention was about 3 times higher than that prepared by the method of Comparative Examples 1 and 2, and the content of Rb1, Rb2, Rc and Rd, which are Panax Diol-based ginseng saponins, the ginseng extract prepared by the method of the present invention was also remarkably higher than that prepared by the method of Comparative Examples 1 and 2.

### Example 2

### Effect of Enhancing Activity of Collagen Promoter in Ginseng Extract

Human normal fibroblast was inoculated in each well of a 6-well microplate containing a DMEM medium at 2×10⁵ cells/well, and cultured at a 5%-CO₂ incubator at 37° C. for 24 hours. After the culture, the medium was taken out from the well, and changed to an FBS-free DMEM. Then, 2 ug of a COL1A2 promoter luciferase reporter DNA was transfected into the human normal fibroblast using Superfect™ (Qiagen, Inc.). After 24 hours from transfection, it was treated with the ginseng extracts prepared in Example 1 at 100 ppm, 500 ppm, and 1000 ppm, respectively, and cultured for 24 hours. Thereafter, the cells were collected, and then luminescence was measured at 450 nm using a Luminometer (Berthold, Germany) (see Planta medica 2005; 71:338-343).

As shown in FIG. 1, it was confirmed that the ginseng extract allowed the activity of the COL1A2 promoter, and increased according to the concentrations. From this result, it was also found that the ginseng extract of Example 1 potentially induces collagen production.

### Example 3

### Effect of Enhancing Collagen Synthesis of Ginseng Extract

Human normal fibroblast was inoculated in each well of a 6-well microplate containing a DMEM medium at 2×10⁵ cells/well, and cultured at a 5%-CO₂ incubator at 37° C. for 24 hours. After the culture, the medium was taken out from the well, and it was treated with the ginseng extracts prepared in Example 1 at 100 ppm, 500 ppm, and 1000 ppm, respectively, and cultured for 24 hours in a 0.05% FBS-containing DMEM. Thereafter, the cell medium was collected to prepare a sample.

For the amount of collagen synthesis, the amount of procollagen type I C-peptide (PICP) was measured using a collagen measuring kit (Procollagen Type I C-peptide EIA kit (MK101), Takara, Kyoto, Japan). After diluting the standard solution contained in the collagen kit, an absorbance was measured at 450 nm to make a standard concentration curve. To each of the wells, was added 100 μl of a POD enzyme-attached antibody solution. The standard solution each diluted according to the concentrations and the supernatant of the sample were mixed in each amount of 20 μl. The mixed solution was left to stand at 37° C. for 3 hours, and the contents were all removed, and each well was washed with 400 μl of PBS four times. To the well, 100 μl of a substrate solution was added, which was left to stand at room temperature for 15 minutes. In order to stop the reaction, 100 μl of a 1 N sulfuric acid solution was added thereto, and mixed, and then an absorbance was measured at 450 nm. These experiments were carried out in duplicate at the same time, and an average was determined therefrom. The amount of PICP was calculated from the standard concentration curve by applying the absorbance of the sample, and determined in terms of ng/2×10⁵ cells.

The results are shown in FIG. 2, and from the results, it was found that the ginseng extract prepared by the preparation method of the present invention had enhanced collagen I synthesis in the human normal fibroblast.

### Example 4

### Effect of Inhibiting Collagenase Activity

Human normal fibroblast was inoculated in each well of a 6-well microplate containing a DMEM medium at 2×10⁵ cells/well, and cultured at a 5%-CO₂ incubator at 37° C. for 24 hours. After the culture, the medium was taken out from the well, and serum-starvation was induced for 16 hours in a 0.05% FBS-containing DMEM. After 16 hours, it was treated with 20 ng/ml of TNF-α alone or in combination with the samples (the ginseng extracts prepared in Example 1 at 100 ppm, 500 ppm, and 1000 ppm, respectively) for 24 hours. Thereafter, the cell medium was collected to prepare a sample.

For the method for measuring the activity level of a collagenase, a collagen decomposing enzyme, an antibody against collagenase was used. In this test, a Type I Collagenase assay kit (Amersham Biosciences, RPN2629) was used, and an absorbance was measured with an ELISA reader. The standard value determined was expressed in mean±standard variation, and a statistical significant was assayed with a t-test using a SPSS/PC+ program. The results are shown in FIG. 3. As shown in FIG. 3, it was found that the ginseng extract of the present invention has an effect of inhibiting collagenase activity.

### Example 5

### Antioxidant Effect

In order to measure the antioxidant effect of the ginseng extract prepared by the method of the present invention, a DPPH (1,1-diphenyl-2-picrylhydrazyl) method was used.

DPPH is a color-developed radical, and can be used to directly confirm the radical removing ability of the same. A sample was dissolved in 4 ml of distilled water or a solvent, 1 ml of 100 μM DPPH was mixed therewith, and incubated at room temperature for 30 minutes. An absorbance was measured at 517 nm to determine the amount of the remaining DPPH. The blank of this test is distilled water or a solvent, and a control is an experimental group composed only of DPPH and distilled water without the sample. The antioxidant ability (Radical Scavenging activity: RSA) was determined by the following equation 1, compared with the absorbances of the controls, and the lowered ratios are shown in Table 2.

RSA (%)=[Absorbance of Hydroxylate/Absorbance of Control]×100 [Equation 1]

TABLE 2
Antioxidant effect of ginseng extract

| | Concentration | | | | | | | |
|---|---|---|---|---|---|---|---|---|
| | of ginseng | | | | | | | |
| | extract (%) | | | | | | | |
| | | | | | | | | Vitamin C |
| | | | | | | | | Vitamin C |
| | 0 | 0.01 | 0.025 | 0.05 | 0.1 | 0.25 | 0.5 | (1 mM) |
| RSA (%) | 100 | 85 | 65 | 45 | 40 | 32 | 28 | 35 |

As shown in Table 2, it was found that the ginseng extract exhibited an ability of removing the radicals generated according to the concentrations.

### Preparative Example

### Cosmetic Composition Containing Ginseng Extract

### 1-1. Cream Containing Ginseng Extract

The ingredients and the contents of a nutrient cream containing the ginseng extract are as shown in the following Table 3. Purified water, triethanol amine, and propylene glycol in an aqueous phase were heated to 70° C. for dissolution, and fatty acids, oily ingredients, an emulsifier and a preservative in an oily phase were heated to 70° C. for dissolution, and both of the obtained solutions were combined for emulsification. After completion of the emulsification, the solution was cooled to 45° C., and the ginseng extract and a flavor were added thereto, dispersed, and then cooled to 30° C.

TABLE 3
Ingredients and contents of nutrient cream containing
ginseng extract

| Ingredients | Contents (wt %) |
|---|---|
| Ginseng extract | 2% or 5.0 or 10% |
| Jojoba oil | 5.0 |
| Liquefied paraffin | 7.0 |
| Cetearyl alcohol | 2.0 |
| Polyglyceryl-3 methyl glucose | 2.0 |
| distearate | |
| Glyceryl stearate | 0.5 |
| Squalane | 3.0 |
| Propylene glycol | 4.0 |
| glycerin | 5.0 |
| Triethanol amine | 0.3 |
| Carboxy vinylpolymer | 0.3 |
| Tocopheryl acetate | 0.2 |
| Preservative, flavor | Trace amount |
| Purified water | Residual amount |
| Total amount | 100 |

### 1-2. Toner Containing Ginseng Extract

### A. Softening Toner

The ingredients and the contents of a softening toner containing the ginseng extract are as shown in the following Table 4. Purified water was added to the aqueous ingredients, and dissolved at room temperature to prepare an aqueous phase. The ginseng extract, the oily ingredients, an emulsifier, a preservative and a flavor were dissolved at room temperature. The obtained phase was combined with the aqueous phase, and two phases were mixed and filtered.

TABLE 4
Ingredients and contents of softening toner containing
ginseng extract

| Ingredients | Contents (wt %) |
|---|---|
| Ginseng extract | 2.00 |
| EDTA | 0.05 |
| PEG 1500 | 4.00 |
| Carbomer | 0.16 |
| 1,3-butylene glycol | 3.00 |
| Polyoxyethylene hydrogenated castor oil | 0.45 |
| Triethanolamine | 0.12 |
| Preservative, flavor, colorant | Trace amount |
| Purified water | Residual amount |
| Total amount | 100 |

### B. Astringent Toner

The ingredients and the contents of an astringent toner containing the ginseng extract are as shown in the following Table 5. Purified water was added to the aqueous ingredients, and dissolved at room temperature to prepare an aqueous phase. The ginseng extract, the oily ingredients, an emulsifier, a preservative and a flavor were dissolved at room temperature with addition of ethanol to prepare an alcohol phase, which was combined with the aqueous phase. The two phases were mixed and filtered.

TABLE 5
Ingredients and contents of astringent toner containing
ginseng extract

| Ingredients | Content (wt %) |
|---|---|
| Ginseng extract | 2.00 |
| Glycerin | 2.00 |
| 1,3-butylene glycol | 3.00 |
| EDTA | 0.05 |
| Ethanol | 7.00 |
| Polyoxyethylene hydrogenated castor oil | 0.40 |
| Preservative, flavor, colorant | Trace amount |
| Purified water | Residual amount |
| Total amount | 100 |

### C. Lotion

The ingredients and the contents of a lotion containing the ginseng extract are as shown in the following Table 6. Purified water, triethanol amine, and butylene glycol in an aqueous phase were heated to 70° C. for dissolution. Then, fatty acids, oily ingredients, an emulsifier, and a preservative in an oily phase were heated to 70° C. for dissolution. The both obtained solutions were combined for emulsification. After completion of the emulsification, a 2% xanthan gum solution as a hydrophilic thickener was added to a concentration of 0.05 wt % based on the total weight. The solution was cooled to 45° C., and the ginseng extract, a flavor, and a colorant were added thereto, mixed, and then cooled to 30° C.

TABLE 6
Ingredients and contents of lotion containing ginseng
extract

| Ingredients | Content (wt %) |
|---|---|
| Ginseng extract | 5.00 |
| Glycerin | 3.00 |
| Carbomer | 0.10 |
| Xanthan gum | 0.05 |
| 1,3-Butylene glycol | 3.00 |
| Polyglycerin-3 methyl glucose | 1.50 |
| distearate | |
| Glycerin stearate | 0.50 |
| Cetearyl alcohol | 0.30 |
| Jojoba oil | 3.00 |
| Liquefied paraffin | 2.00 |
| Squalane | 3.00 |
| Dimethicone | 0.50 |
| Tocopheryl acetate | 0.20 |
| Triethanolamine | 0.10 |
| Preservative, Flavoring agent, | Trace amount |
| Colorant | |
| Purified water | Residual amount |
| Total amount | 100 |

### D. Gel

The ingredients and the contents of a gel containing the ginseng extract are as shown in the following Table 7.

TABLE 7
Ingredients and contents of gel containing ginseng extract

| Ingredients | Content (wt %) |
|---|---|
| Ginseng extract | 2.50 |
| EDTA | 0.02 |
| 1,3-Butylene glycol | 4.00 |
| Carbomer | 0.60 |
| Glycerin | 5.00 |
| Hydroxyethyl cellulose | 0.15 |
| Triethanolamine | 0.50 |
| Trichloric acid | 0.20 |
| Ethanol | 5.00 |
| Polyoxyethylene hydrogenated castor oil | 4.00 |
| Preservative, Flavoring agent, Colorant | Trace amount |
| Purified water | Trace amount |
| Total amount | 100 |

### E. Essence

The ingredients and the contents of an essence containing the ginseng extract are as shown in the following Table 8. A thickening agent was slowly added to purified water while stirring, and uniformly dispersed therein. The aqueous ingredients were mixed to prepare an aqueous phase. Then, an emulsifier, a skin softener, a flavor and a preservative were added with ethanol, and dissolved. The alcohol phase was added to the aqueous phase while stirring, and then an alkalifying agent was added thereto. Finally, the ginseng extract and a colorant were added.

TABLE 8
Ingredients and contents of essence containing ginseng
extract

| Ingredients | Content (wt %) |
|---|---|
| Ginseng extract | 10.00 |
| EDTA | 0.05 |
| Potassium-cetylphosphate | 0.30 |
| Glycerin | 5.00 |
| 1,3 butylene glycol | 4.00 |
| Glyceryl stearate/PEG-100 stearate | 0.50 |
| Sorbitan stearate | 0.30 |
| Jojoba oil | 0.50 |
| Macadamia nut oil | 0.50 |
| Tocopheryl acetate | 0.50 |
| Butylated hydroxytoluene | 0.05 |
| Dimethicone | 3.00 |
| Polyacrylamide/C13–14 isoparaffin/laureth-7 | 1.50 |
| Glyceryl polymethacrylate/propylene glycol | 5.00 |
| Ethanol | 3.00 |
| Preservative, flavor, colorant | Trace amount |
| Purified water | Residual amount |
| Total amount | 100 |

### F. Pack

The ingredients and the contents of a pack containing the ginseng extract are as shown in the following Table 9. Purified water was heated to 70° C., and a film forming agent and a moisturizer were added and dissolved to obtain a clear aqueous phase. Then, the ginseng extract, an emulsifier, a preservative, and a flavor were dissolved in alcohol, and this phase was combined with the aqueous phase. The combination was cooled to 30° C. to prepare a pack.

TABLE 9
Ingredients and contents of pack containing ginseng extract

| Ingredients | Content (wt %) |
|---|---|
| Ginseng extract | 5.00 |
| Benzophenone-5 | 0.10 |
| 1,3-Butylene glycol | 5.00 |
| Carbomer | 1.00 |
| Triethanolamine | 0.85 |
| Glycerin | 4.00 |
| Alantoin | 0.10 |
| DL-Panthenol | 0.50 |
| Preservative, flavor, colorant | Trace amount |
| Purified water | Residual amount |
| Total amount | 100 |

### Comparative Preparative Example

### Cream Containing Purified Water

The ingredients and the contents of the cream having the ginseng extract replaced by purified water in the preparation process of Preparative Example 1-1 are shown in the following Table 10.

TABLE 10

| Ingredients | Content (wt %) |
|---|---|
| Jojoba oil | 5.0 |
| Liquefied paraffin | 7.0 |
| Cetearyl alcohol | 2.0 |
| Polyglyceryl-3 methyl glucose distearate | 2.0 |
| Glyceryl stearate | 0.5 |
| Squalane | 3.0 |
| Propylene glycol | 4.0 |
| Glycerin | 5.0 |
| Triethanol amine | 0.3 |
| Carboxy vinylpolymer | 0.3 |
| Tocopheryl acetate | 0.2 |
| Preservative, flavor | Trace amount |
| Purified water | Residual amount |
| Total amount | 100 |

### Example 6

### Experiment of Confirmation of Safety of Ginseng Extract on Skin of Human Body

In order to confirm safety of the ginseng extract on the skin of a human body, an experiment to rectify skin safety was carried out. As the test method, a skin cumulative stimulation test was carried out.

Formulations having squalene as a base with addition of the ginseng extracts at 1%, 5%, and 10% were prepared. Each of the formulations was applied in cumulative patches to forearm areas of 30 healthy adults in total 9 times for 24 hours every second day (total amount), and it was determined whether the ginseng extract stimulates the skin or not. For the method for applying the patches, a Finn chamber (Epitest Ltd, Republic of Finland) was used. 15 ul-portions of the skin external agents were dropped on the chamber, and patches were applied thereon. The response level of the skin per every application was scored using the following equation 2. The results are shown in the following Table 11.

Mean response level=[[Response index×Reaction level/Number of subject to be test×Highest score (Score 4)]×100]/Number of conducted tests (9) [Equation 2]

Here, regarding the response levels, means±score, +means 2 scores, and ++means 4 scores. If the mean response level is less than 3, the composition was considered to be safe on the skin.

TABLE 11

| | Number of persons to exhibit response | | | | | | | | | | | | | | | | | | | | | | | | | | | Mean |
|---|---|---|---|---|---|---|---|---|---|---|---|---|---|---|---|---|---|---|---|---|---|---|---|---|---|---|---|---|
| | Week 1 | | | | | | | | | Week 2 | | | | | | | | | Week 3 | | | | | | | | | Re- |
| Test | 1^{st} | | | 2^{nd} | | | 3^{rd} | | | 4^{th} | | | 5^{th} | | | 6^{th} | | | 7^{th} | | | 8^{th} | | | 9^{th} | | | sponse |
| Materials | ± | + | ++ | ± | + | ++ | ± | + | ++ | ± | + | ++ | ± | + | ++ | ± | + | ++ | ± | + | ++ | ± | + | ++ | ± | + | ++ | level |
| Control | 2 | — | — | 0 | — | — | — | — | — | — | — | — | — | — | — | — | — | — | — | — | — | — | — | — | — | — | — | 0.18 |
| (Squalene) | | | | | | | | | | | | | | | | | | | | | | | | | | | | |
| Ginseng | 2 | — | — | 0 | — | — | — | — | — | — | — | — | — | — | — | — | — | — | — | — | — | — | — | — | — | — | — | 0.18 |
| extract | | | | | | | | | | | | | | | | | | | | | | | | | | | | |
| (1%) | | | | | | | | | | | | | | | | | | | | | | | | | | | | |
| [Ex- | | | | | | | | | | | | | | | | | | | | | | | | | | | | |
| perimental | | | | | | | | | | | | | | | | | | | | | | | | | | | | |
| group 1] | | | | | | | | | | | | | | | | | | | | | | | | | | | | |
| Ginseng | 2 | — | — | 0 | — | — | — | — | — | — | — | — | — | — | — | — | — | — | — | — | — | — | — | — | — | — | — | 0.18 |
| extract | | | | | | | | | | | | | | | | | | | | | | | | | | | | |
| (5%) | | | | | | | | | | | | | | | | | | | | | | | | | | | | |
| [Ex- | | | | | | | | | | | | | | | | | | | | | | | | | | | | |
| perimental | | | | | | | | | | | | | | | | | | | | | | | | | | | | |
| group 2] | | | | | | | | | | | | | | | | | | | | | | | | | | | | |
| Ginseng | 2 | — | — | 0 | — | — | — | — | — | — | — | — | — | — | — | — | — | — | — | — | — | — | — | — | — | — | — | 0.18 |
| extract | | | | | | | | | | | | | | | | | | | | | | | | | | | | |
| (10%) | | | | | | | | | | | | | | | | | | | | | | | | | | | | |
| [Ex- | | | | | | | | | | | | | | | | | | | | | | | | | | | | |
| perimental | | | | | | | | | | | | | | | | | | | | | | | | | | | | |
| group 3] | | | | | | | | | | | | | | | | | | | | | | | | | | | | |

| | | | | | | | | | |
|---|---|---|---|---|---|---|---|---|---|
| Number | 30 | 30 | 30 | 30 | 30 | 30 | 30 | 30 | 30 |
| of | | | | | | | | | |
| persons | | | | | | | | | |
| to be | | | | | | | | | |
| test | | | | | | | | | |

As shown in Table 11, in any of the experimental groups 1, 2 and 3, the number of the persons having ±, + and ++were 2, 0, and 0, respectively. The mean response level was 0.18, which is less than 3, and thus the composition was found to be safe. Therefore, the ginseng extract prepared by the method of the present invention did not exhibit remarkable cumulative stimulation, and it was found to be safe on the skin of a human body.

### Example 7

### Measurement of Effect of Cosmetics Containing Ginseng Extract to Improve Skin Wrinkles

The effect of a cosmetic containing the ginseng extract to improve skin wrinkles was measured by a clinical test. The nutrient creams of Preparative Example 1-1 (nutrient creams having the ginseng extracts at 2%, 5% and 10%) and of Comparative Preparative Example (nutrient cream containing purified water) were used. The effect of improving skin wrinkles was evaluated by measuring the changes in skin resilience. Measurement on skin resilience was made at a temperature of 24 to 26° C., and a humidity maintained at 38 to 40%, with 30 healthy women (25 to 35-year-old) as the subjects to be test, by applying three kinds of the nutrient creams of Preparative Example 1-1, and the nutrient cream of Comparative Preparative Example on the face of the subject twice per day for 3 months, and then measuring the skin resilience using Cutometer SEM 474. The evaluation standards were relatively set as follows: Score 0: No skin resilience through Score, 5: A lot skin resilience. The results are shown in Table 12.

TABLE 12
Effect of ginseng extract on improving skin resilience

| | Skin |
|---|---|
| Test items | resilience |
| Preparative Example 1-1 (Cream | 4.1 |
| containing ginseng extract at 2%) | |
| Preparative Example 1-1 (Cream | 4.5 |
| containing ginseng extract at 5%) | |
| Preparative Example 1-1 (Cream | 4.8 |
| containing ginseng extract at 10%) | |
| Comparative Preparative Example | 1.45 |
| ((Cream containing ginseng extract | |
| at 0%) | |

As shown in Table 12, it was found that the cream containing the ginseng extract of Preparative Example 1-1 of the present invention was better in the effect of improving skin wrinkles, as compared with the cream of Comparative Preparative Example, and further, it was suggested that since skin resilience is further improved as the concentration of the ginseng extract is increased, the ginseng extract exhibits an effect of improving skin wrinkles.

### EFFECTS OF THE INVENTION

By the simpler process according to the present invention, the total content of the ginsenocides can be remarkably increased, while maintaining a high content of Rb1, Rb2, Rc and Rd, which are the effective ingredients of ginseng. Further, the ginseng extract extracted by the process according to the present invention promotes the collagen formation, resulting in the effect of improving skin wrinkles. Furthermore, it has substantially no stimulation on the skin, whereby it can be used as a new raw material for improving skin wrinkles.

What is claimed is:

1. A method for preparing a ginseng extract having an increased content of physiologically active ingredients, comprising the steps of steaming ginseng at 95 to 100° C. for 1 to 24 hours, collecting the generated vapor by cooling, and repeating the process twice.

2. The method according to claim 1, wherein the ginseng is Fresh ginseng.

3. The method according to claim 1, wherein the physiologically active ingredients are Rb1, Rb2, Rc and Rd.

4. A composition for promoting collagen synthesis, comprising the ginseng extract prepared by the method according to claim 1.

5. The composition for promoting collagen synthesis according to claim 4, wherein the ginseng extract is contained in a proportion of 0.001 to 20.0 w/w, based on the total dry weight of the composition.

6. A cosmetic product for improving skin wrinkles, comprising the composition according to claim 4.

7. The cosmetic product according to claim 6, wherein the cosmetic product for improving skin wrinkles is a nutrient cream, an astringent toner, a softening toner, an essence, a nutrient gel, or a massage cream.
